(19) United States
(12) Patent Application Publication
(10) Pub. No.: US 2016/0346239 A1
(43) Pub. Date: Dec. 1, 2016

(54) HEMOSTATIC COMPOSITION AND DEVICE

(71) Applicant: Maxim Korobov, Moscow (RU)

(72) Inventor: Maxim Korobov, Moscow (RU)

(21) Appl. No.: 15/138,562

(22) Filed: Apr. 26, 2016

Related U.S. Application Data
(60) Provisional application No. 62/153,442, filed on Apr. 27, 2015.

(51) Int. Cl. A61K 31/195; A61K 45/06; A61K 47/26; A61K 47/02; A61K 47/36; A61K 47/18

(52) U.S. Cl. CPC A61K 31/195; A61K 47/36; A61K 47/18; A61K 47/26; A61K 47/02; A61K 45/06

(57) ABSTRACT

A hemostatic composition comprises calcium alginate, a chitosan, epsilon-aminocaproic acid, an acid selected from aminomethylbenzoic acid and tranexamic acid, and tannin. The method of making the hemostatic composition comprises mixing one or more polysaccharide bases, tannin, a fibrinolytic inhibitor, colloidal silver and a solvent to form a mixture, and drying the mixture at a temperature between 25° C. and 80° C. until residual moisture content is approximately 15 to 20%.

### CROSS-REFERENCE TO RELATED APPLICATIONS

This application claims the benefit of U.S. Provisional Patent Application No. 62/153,442 filed on Apr. 27, 2015, the contents of which are incorporated herein in their entirety.

### FIELD AND BACKGROUND OF THE INVENTION

The present invention relates to a hemostatic composition which comprises biologically absorbable ingredients. In one aspect, the invention is for a local hemostatic device, such as for arresting the flow of blood, and which may be applied in first aid and emergency care on or to bleeding wounds and damaged blood vessels to stop capillary or mixed bleedings of various intensity. It may also be used in surgical practice. In one aspect, the hemostatic composition and device of the invention may be used by covering or dumping or relocating the powder composition of the invention directly on the source of bleeding into the wound.

If the blood vessels are damaged by a physical trauma, including the consequences of surgery, a bleeding occurs. Depending on the amount of damage, bleeding may lead to blood loss, which may affect the normal function of an individual. In cases of bleedings originating in the osseous non-expandable cavities, the accumulation of extravasated blood may cause soft tissue become damaged due to elevated pressure. If the bleeding is left alone and not treated, it will eventually be arrested by a normally occurring physiological process characterized by a chain of events involving combined activity of vascular, platelet, and plasma factors.

### SUMMARY OF THE INVENTION

According to one aspect of the invention, there is provided a hemostatic composition comprising: calcium alginate; a chitosan; epsilon-aminocaproic acid; an acid selected from aminomethylbenzoic acid and tranexamic acid; and tannin.

According to a further aspect of the invention, there is provided a hemostatic composition comprising: a polysaccharide base; a fibrinolytic inhibitor; and tannin.

According to yet a further aspect of the invention, there is provided a method of making a hemostatic composition, the method comprising: mixing one or more polysaccharide bases, tannin, a fibrinolytic inhibitor, colloidal silver and a solvent to form a mixture; and drying the mixture at a temperature between 25° C. and 80° C. until residual moisture content is approximately 15 to 20%.

The composition and device of the invention is in one embodiment thereof soft and a has a powdered or granular presentation. The particle size can vary widely, from visible to the naked eye to microscopic in size.

Preferably, both the composition and device of the invention do not cause any damaging or significant damaging effect on the surrounding tissue, has no thermal impact during application (it practically does not heat up), and is not toxic. The powder of the composition readily repeats the wound profile and tightly covers it.

The composition of the present invention can be used for enhancing the arrest of bleeding in a number of instances of surgical interventions or other injuries. This includes surgeries of large abdominal organs (liver, spleen, or intestines); neurosurgery to prevent pressure damages of the cerebral or nerve tissues; orthopedic surgery during which extensive hemorrhages frequently occur which are difficult to arrest by other means; in vascular surgery to arrest seeping bleedings from the sites of suturing; oral or dental surgery such as extraction of teeth; and nose-bleeding.

In vitro experiments revealed hemostatic properties of the composition, which were proved by the experiments on arresting parenchymal bleedings from liver and spleen wounds, as well as wounds of various etiologies without causing any reaction in the surrounding tissues.

Also, the composition was found to produce a stimulating effect on regenerative processes in the wound, which is reflected in the acceleration of granulation tissue growth, promotion of marginal and insular epithelization, and the facilitation of favorable conditions for epithelial cell migration, vascular invasion and scar-free wound healing.

One of the hemostatic effects (or type of action) of the sorbent-based composition and devices of the invention may be associated with the porosity and ability to absorb blood. For example, a typical gelatin sponge absorbs the amount of blood about 40-50 times higher than its own weight. However, sponge swelling may lead to adverse events. Swelling after they have absorbed liquid, sponges may compress the surrounding tissues, and there occurs a possibility of nerve damage. Therefore, there is a need to address the problem of limiting the swelling of hemostatic devices comprising sorbents, which swell after contact with bodily fluids. This problem is preferably significantly addressed when creating or using a hemostatic composition of the present invention.

One advantage of the present invention is to provide a device ensuring pronounced hemostatic properties with reduced tendency to swell, making it safer to use, as well as having reparative and antiseptic effects.

This may be achieved by virtue of the composition, which in one aspect of the invention comprises a polysaccharide base including calcium alginate and chitosan; tannin (tannic acid), or other organic tannins, or tannin-containing plant extracts; fibrinolytic inhibitors (e.g. epsilon-aminocaproic, tranexamic, and aminomethylbenzoic acid); and colloidal silver.

The polysaccharide base of the composition may be cross-linked. Cross-linking using pharmaceutically acceptable chemical agents is possible as well as using radiation; the most preferable crosslinking method is carried out via a dehydrothermal treatment method.

The degree of crosslinking of the composition polymer base has an effect on some of its functional properties, including the ability to adhere to the wound tissues, absorption of surrounding biological fluids, etc. The degree of crosslinking of the polymer base can be controlled. The most preferred degree of swelling (composition volume increase upon contact with bodily fluids) ranges from 1.5 to 5 times.

The swelling properties of the composition can be measured by the increase in the composition volume in a graduated cylinder filled with distilled water. The amount of the device placed in the cylinder is measured, and then and excess of water is added. Then, the increased volume of the device after the end of swelling is measured. Also, the swelling can be measured in the normal saline, which gives a more accurate estimation of swelling in bodily fluids.

The hemostatic composition of the present invention may contain a certain amount of residual water, such as up to about 8% (w/w). However, the residual water is not calculated as part of the total weight of the sponge. This means that when it comes to the sponge comprising a certain % wt. of a certain component (such as calcium alginate), the % wt. is calculated based on the anhydrous basis of the total weight of the composition, i.e. based on its total weight excluding any water which may be bound.

### DETAILED DESCRIPTION OF THE INVENTION

Further details of the invention including embodiments and variations thereof will be described below.

Possible Contents of Hemostatic Composition

The active base of the composition is comprised of natural polysaccharides. These include calcium alginate, which is an anionic polysaccharide, and chitosan, which is a cationic polysaccharide. They ensure or facilitate blood absorbance and concentration of blood components involved in hemostasis on particle surfaces of the device; moreover, their ratio optimizes the hemostatic properties of the composition.

Other alginates may be used in the hemostatic composition. It is preferable to choose the multivalent metal ions such that they form slightly soluble compounds with alginate, i.e. act as cross-linking metal ions. Such polyvalent metal ions include, for example, ions of alkaline earth metals and transition metals that form slightly soluble compounds with alginates. Alkaline earth metal ions, such as magnesium or calcium, are preferred. Calcium is particularly preferred. Therefore, according to the present invention, calcium salts are most preferred, since they are physiologically compatible and have a high capacity to cross-linking or gel formation in relation to alginates. Furthermore, calcium alginate delivers calcium ions (Ca²⁺) to the wound area and, thereby, activates the hemostatic process and promotes healing in the wound area during the subsequent processes. In addition to chitosan, the composition may include its derivatives, for example, salts, such as chitosan chloride, chitosan acetate, chitosan citrate, as well as its pharmaceutically acceptable modifications. The use of these derivatives does not limit the use of other derivatives in the composition of the present invention.

Fibrinolytic inhibitors included in the composition improve its hemostatic properties. The mammalian body has an inherent fibrinolytic system which is activated by the deposition of fibrin. By dissolving fibrin, this system helps to maintain the lumen of the damaged blood vessel open. However, in situations where the goal is a rapid hemostasis, fibrinolytic activity may counteract the hemostatic effect of a hemostatic additional device, such as the composition according to the present invention. Pharmacological pathways of fibrinolytic inhibition may involve inhibiting plasmin or plasminogen-to-plasmin transition. Application of fibrinolytic inhibitors reduces the influence of the proteolytic enzyme plasmin on the cleavage (by hydrolysis) of soluble peptides from fibrin, which are subsequently cleaved by peptidases. It is known that plasmin also acts on other coagulation factors: fibrinogen (Factor I), Factor V, Factor VIII, Factor XII, and prothrombin, which empowers it not only with thrombolytic, but also anticoagulant effect. Thus, due to fibrinolytic inhibitors, plasmin activity or its plasma level decreases, leading to increased clotting. Accordingly, the agents with anti-fibrinolytic properties included in the hemostatic composition of the present invention may be advantageous. The composition may primarily include anti-fibrinolytic agents selected from the group consisting of aminocaproic, tranexamic, and aminomethylbenzoic acids. The most preferable is the combination of anti-fibrinolytic agents, aminocaproic and tranexamic acids, or aminocaproic and aminomethylbenzoic acids. When applied topically, the combinations of these agents significantly reduce the risk of re-bleeding for significantly longer time period than each of these agents alone. Application of these fibrinolytic inhibitors does not limit the possibility of application of other similar agents with similar action in the hemostatic composition of the present invention. When determining the weight percent in the composition, the following agents may be applied: aprotinin, pepstatin, leupeptin, antipain, chymostatin, fibronectin, and other means.

Tannin or other organic tannins enhance hemostatic activity of the composition and ensure reduced tissue permeability, thereby decreasing the risk of re-bleeding. Tannins denature cell proteins, forming an albuminate protective film and, also, have bactericidal or bacteriostatic effect on some microorganisms. Tannins are colloid substances with negatively charged particles and dehydrating properties. It is known that the presence of a large number of hydroxyl groups in tannins increase the activity of hemostatic processes. The present invention in one aspect may utilize organic tannins as polyphenolic compounds with a molecular weight from 500 to 20,000 mainly derived from pyrogallol, catechol, phloroglucinol, which are capable of forming strong bonds with proteins and alkaloids and have tanning properties. Their non-limiting examples include: gallic acid, ellagic acid, catechol, pyrogallol, and phloroglucinol. The hemostatic composition of the present invention may also use tannin-containing plant extracts, for example, the extracts of oak, bergenia (Bergenia crassifolia), lagohilusa (Lagochilus inebrians Bunge), smoke tree (Cotinus coggygria), and sumach (Rhus coriaria). Application of the said extracts does not limit the use of other tannin-containing extracts. It is preferable to use plant tannins or synthetic tannins in the composition of the present invention, since chemically they are quite pure and easily standardized, which is important for mass production of the device.

One of the consequences of bleeding is that there may be a very high chance of microorganism infection of skin and other traumatic lesions. Thus, it may be advantageous to introduce an antiseptic component into the composition. Colloidal silver particles can be introduced into the composition. Silver nanoparticles are atomic silver agglomerates with the size of 1-100 nm, the surface of which is surrounded by a layer of stabilizer molecules, which ensures a long life-time of the water/stabilizer/silver nanoparticles system. Preferably, the particle size should be within the range 3-30 nm. A method for producing reconstituted silver particles is not significant if it is pharmaceutically acceptable. The approach preferred for this invention is a method for recovering silver nitrate in an aqueous or aqueous-alcoholic solution of tannins based on tannin.

Among the excipients, various substances regulating and maintaining a certain level of acidity may be used both during the technical process and determining the pH value during the application of the device. For example, the following acids may be employed: hydrochloric acid, acetic acid, citric acid, succinic acid, and lactic acid. The hydroxides are: sodium hydroxide, potassium hydroxide, calcium hydroxide, barium hydroxide and magnesium hydroxide.

Also, the hemostatic device of the present invention may contain a buffering agent. Examples of buffering agents include alkali salts, such as: chlorides, acetates, citrates, phosphates, hydrogen phosphates, carbonates, bicarbonates, and succinates. Obviously, it is possible to use mixtures of the abovementioned buffering agents. The use of these substances does not limit the use of other similar substances.

### Examples of Preferred Composition

In order to ensure the hemostatic properties of the composition of the present invention, the important ingredients are:

(1) calcium alginate;

(2) chitosan;

(3) epsilon-aminocaproic acid;

(4) aminomethylbenzoic acid or tranexamic acid (an alternative, but fully functioned option);

(5) tannin (tannic acid), or other organic tannins, or tannin-containing plant extracts.

Colloidal silver particles are an extra ingredient supplying the composition with antiseptic activity, which expands the scope of its application.

Excipients may include various agents that regulate and maintain certain level of acidity both during the technical process and determining the pH value during the application of the device.

The following table sets out the various ingredients or components of one aspect of the hemostatic composition in accordance with the present invention, including a series of ranges in which such component may be present, the ranges varying from effective functionality of the components to preferred functionality thereof.

Composition ingredient ratios, in weight % (on the dried basis) are set forth as follows, as examples of the invention:

| | For effective | For special | |
|---|---|---|---|
| Components | action | effectiveness | Optimal effect |
| Calcium | 20-75 | 35-72 | 45-70 |
| alginate | | | |
| Chitosan | 1-7.5 | 1.5-7 | 1.5-6 |
| Epsilon- | 2.5-10 | 3-9 | 4-7 |
| aminocaproic | | | |
| acid | | | |
| Tranexamic acid | 0.5-2.5 | 0.75-2 | 1-1.5 |
| Aminomethylbenzoic | 0.5-3 | 1-2.5 | 1-2 |
| acid | | | |
| Tannin, or | 1-30 | 2.5-27 | 4-25 |
| other organic | | | |
| tannins, or | | | |
| tannin- | | | |
| containing | | | |
| plant extracts | | | |
| Colloidal | 1-500 ug/g | 10-450 ug/g | 25-430 ug/g |
| silver | (μg/g) | (μg/g) | (μg/g) |

### Examples of Methods of Preparation

The hemostatic composition in accordance with the invention may be prepared in a number of different manners, and in a preferred embodiment, the preparation may comprise the following steps:

(I) Mixing the polysaccharide base of the composition, tannin (or other organic tannins, including tannin-containing plant extracts), fibrinolytic inhibitors (epsilon-aminocaproic, tranexamic and aminomethylbenzoic acids) colloidal silver and solvent.

In order to avoid obtaining an inhomogeneous mixture during mixing, which is potentially possible, and to ensure activation of the composition ingredients, mixing in accordance with one aspect of the invention is carried out at an elevated temperature such as a temperature ranging from 25° C. to 80° C., e.g. ranging from 25° C. to 40° C., or within the range from 35° C. to 80° C.

Mixing the composition ingredients and the solvent is typically performed under mechanical influence. Thus, during and/or immediately after mixing, the resulting mixture should preferably be subjected to centrifugation, vortexing at high speed, beating, or other type of mechanical influence, known to the specialist in this field.

(II) Drying of the mixture obtained at the step (I);

After mixing, the resulting mixture can be poured into appropriate trays or placed on Teflon sheets or silicon sheets and dried without stirring. The drying process can be carried about at a temperature from 25° C. to 80° C. until the residual moisture content of 15-20%. Under such conditions, drying is time-consuming. The duration depends on the amount of solvent present in the mixture and the evaporation area and may, in particular, vary from about 12 hours to about 48 hours. Drying the product to the residual humidity of less than 8% can occur at the temperatures of up to 105° C.-115° C., but for not more than 1-2 hours.

Drying under vacuum is more preferable, in which case it is feasible to use the vacuum level of at least about 300 millimeters of mercury (mm Hg). The lower limit is determined by the type of equipment used. However, intense foaming and boiling of the mixture that is being dried should be avoided, especially in the presence of excess solvent therein. Drying temperature can be the same as in drying with no vacuum.

Mixture freeze-drying is even more preferable. Herein, the step of freezing should occur at the temperature of not higher than minus 25° C. During freeze-drying, the process should be carried out to the residual moisture content of not more than 5%.

(III) Optional thermal treatment of the mixture obtained at the step (II).

After the step (II) above, the product may be optionally stabilized by the treatment at elevated temperatures, such as within the range of about 110° C. to 180° C. Stabilization time depends on the temperature, but it usually ranges from about 15 minutes to 2 hours. This step is advantageously carried out if no high-temperature regime was used at the step (II). It is preferable to carry out stabilization under vacuum.

(IV) Milling the product obtained at the steps (II) and (III).

If the product, obtained in accordance with these steps as set forth above, is not a powder or granules of the desired fractional composition according to the present invention, it may be subjected to milling. Thus, during drying on trays or sheets, a sponge-like form of the product may form. Also, clumps and agglomerates (particles caked into lumps) may form during drying. It is possible to mill the product into powder, for example, using a rotary bed, extrusion, crusher or centrifugal mill. It is preferable to mill the product on a grinding mill with grind setting adjustment. If drying is carried out on the equipment which results in the product as a powder or granules of the desired fraction size, then milling may not be necessary. The average particle size of the present invention is less than 250 um. More preferable average particle size ranges from 70 to 100 um.

It should be kept in mind that the fractional composition of the powder particles should be a compromise between average sized particles, large particles (250 um to 500 um) and smaller particles of at least 10 um. On the one hand, the presence of a large amount of fine fractions in the powder significantly increases its active surface area, particle adhesion to the wound surface and reduces the time of bioresolution (bioabsoprtion) of the composition. On the other hand, smaller particles are more readily washed with seeping blood, and insufficient amounts of large particles in the fractional composition of the powder may lead to insufficient wound drainage. As a result, the composition may be poorly soaked with blood when the applied layer thickness is considerable; consequently, a thin and fragile blood clot forms in the wound area.

(V) Filling and packaging of the hemostatic composition.

Individual packages may have various presentations. One preferred package presentation is a sachet or a stick. The preferred weight of the device in the package for pharmacies: 2.5 g and 5 g; for individual first aid kits and surgical use: 5 g and 10 g.

(VI) Final sterilization.

The hemostatic device is sterilized in the package by radiation. Sterilization can be carried out by application of radiation, such as beta- or gamma-radiation. The radiation dose typically lies within the range of 10-60 kGy, such as 20-60 kGy, or 25-50 kGy, or 15-25 kGy, or 15-20 kGy, viz. around 15 kGy, 20 kGy or 25 kGy. Such treatment reduces the bioburden of the device and may also enhance the cross-linking of the molecular chains in the medicinal product. It is preferable to perform sterilization using electron accelerators.

The hemostatic composition and device of the invention may have an individual use on open wounds, or an application in the field, and in traumatic injuries.

For individual use, the device does not require any special professional skills or special storage conditions (from −5° C. to 40° C.), making it easy to use it on wounds of various etiology associated with blood loss.

Application of the device on the open wound surface for torn or stab wounds or various types of bleeding injuries may provide the fast and reliable arrest of bleeding. The composition may be applied so that the target site, for example, the wound, is completely covered.

For strong bleedings, the device is fixed with a bandage and applied in a thick layer (in excess), which facilitates the removal of the retentive bandage without damaging the blood clot, as the upper layer of the medicinal product in contact with the bandage remains dry.

In surgical practice during interventions: additional or complete arrest of the bleeding during surgical interventions on internal organs; treatment of seeping sutures.

The hemostatic device may be primarily used as a powder or in the form of granules; however, in other embodiments of the invention, it is feasible to use in the form of a paste. In the given context, the term “paste” refers to a solid or semi-solid disperse system where the particles of the device powder are dispersed in a liquid medium. The powder of the device may also be referred to as a gel- or paste-forming agent. The paste is characterized by the dynamic viscosity above that of water.

The paste can be prepared by suspending particles of the hemostatic device in a liquid medium, viz. aqueous medium. Usually, about 1-5 ml of liquid medium is used per 1 g of the device. The liquid medium is preferably an aqueous medium, but other acceptable pharmaceutical media can be used. The aqueous medium may contain salts such as sodium chloride, dissolved therein, at the concentrations close to the saline solution.

The device can be used in individual first aid kits, as well as sold through pharmacies.

Throughout this description, the embodiments and examples shown should be considered as exemplars, rather than limitations on the apparatus and procedures disclosed or claimed. Although many of the examples presented herein involve specific combinations of method acts or system elements, it should be understood that those acts and those elements may be combined in other ways to accomplish the same objectives. Acts, elements and features discussed only in connection with one embodiment are not intended to be excluded from a similar role in other embodiments.

As used herein, “plurality” means two or more. As used herein, a “set” of items may include one or more of such items. As used herein, whether in the written description or the claims, the terms “comprising”, “including”, “carrying”, “having”, “containing”, “involving”, and the like are to be understood to be open-ended, i.e., to mean including but not limited to. Only the transitional phrases “consisting of” and “consisting essentially of”, respectively, are closed or semi-closed transitional phrases with respect to claims. Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. As used herein, “and/or” means that the listed items are alternatives, but the alternatives also include any combination of the listed items.

1. A hemostatic composition comprising:
calcium alginate;
a chitosan;
epsilon-aminocaproic acid;
an acid selected from aminomethylbenzoic acid and tranexamic acid; and
tannin.

2. A hemostatic composition as claimed in claim 1 wherein:
the calcium alginate comprises 20 to 75 weight percent;
the chitosan comprises 1 to 7.5 weight percent;
the epsilon-aminocaproic acid comprises 2.5 to 10 weight percent;
the aminomethylbenzoic acid comprises 0.5 to 3 weight percent and tranexamic acid comprises 0.5 to 2.5 weight percent; and
the tannin comprises 1 to 30 weight percent.

3. A hemostatic composition as claimed in claim 2 wherein:
the calcium alginate comprises 35 to 72 weight percent;
the chitosan comprises 1.5 to 7 weight percent;
the epsilon-aminocaproic acid comprises 3 to 9 weight percent;
the aminomethylbenzoic acid comprises 1 to 2.5 weight percent and tranexamic acid comprises 0.75 to 2 weight percent; and
the tannin comprises 2.5 to 27 weight percent.

4. A hemostatic composition as claimed in claim 3 wherein:
the calcium alginate comprises 45 to 70 weight percent;
the chitosan comprises 1.5 to 6 weight percent;
the epsilon-aminocaproic acid comprises 4 to 7 weight percent;
the aminomethylbenzoic acid comprises 1 to 2 weight percent and tranexamic acid comprises 1 to 1.5 weight percent; and
the tannin comprises 4 to 25 weight percent.

5. A hemostatic composition as claimed in claim 1 further comprising colloidal silver.

6. A hemostatic composition as claimed in claim 1 wherein the tannin is selected from one or more of tannic acid, organic tannins, and tannin-containing plant extracts.

7. A hemostatic composition as claimed in claim 1 further comprising residual water.

8. A hemostatic composition as claimed in claim 7 wherein water may be present up to about 8% by weight.

9. A hemostatic composition as claimed in claim 1 further comprising an antiseptic component.

10. A hemostatic composition as claimed in claim 1 further comprising at least one substance for regulating and maintaining a selected level of acidity so as to establish a pH value of the composition.

11. A hemostatic composition as claimed in claim 10 wherein the substance for regulating is selected from one or more of hydrochloric acid, acetic acid, citric acid, succinic acid, lactic acid, sodium hydroxide, potassium hydroxide, calcium hydroxide, barium hydroxide and magnesium hydroxide.

12. A hemostatic composition as claimed in claim 1 further comprising a buffering agent.

13. A hemostatic composition comprising:
a polysaccharide base;
a fibrinolytic inhibitor; and
tannin.

14. A hemostatic composition as claimed in claim 13 wherein the polysaccharide base comprises an anionic polysaccharide and a cationic polysaccharide.

15. A hemostatic composition as claimed in claim 14 wherein the anionic polysaccharide comprises calcium alginate and the cationic polysaccharide comprises chitosan.

16. A hemostatic composition as claimed in claim 15 wherein the chitosan is selected from one or more of chitosan citrate, chitosan chloride, chitosan acetate.

17. A hemostatic composition as claimed in claim 13 wherein the fibrinolytic inhibitor comprises one or more selected from epsilon-aminocaproic acid, aminomethylbenzoic acid and tranexamic acid.

18. A hemostatic composition as claimed in 13 further comprising colloidal silver.

19. A hemostatic composition as claimed in claim 13 wherein the polysaccharide base is cross-linked.

20. A method of making a hemostatic composition, the method comprising:
mixing one or more polysaccharide bases, tannin, a fibrinolytic inhibitor, colloidal silver and a solvent to form a mixture; and
drying the mixture at a temperature between 25° C. and 80° C. until residual moisture content is approximately 15 to 20%.

21. A method as claimed in claim 20 further comprising thermal treatment of the mixture after it has been dried to stabilize the mixture.

22. A method as claimed in claim 20 wherein the mixing is carried out at an elevated temperature of between 25° C. and 80° C.

23. A method as claimed in claim 20 wherein the mixing is carried out with centrifugation, vortexing at high speed, or beating.

24. A method as claimed in claim 20 wherein the drying step is carried out in a vacuum.

25. A method as claimed in claim 20 further comprising the step of milling.

26. A method as claimed in claim 20 further comprising the step of filling and packaging the hemostatic composition.

27. A method as claimed in claim 20 further comprising the step of sterilizing the composition by radiation.
